(19) United States
(12) Patent Application Publication
(10) Pub. No.: US 2007/0043379 A1
(43) Pub. Date: Feb. 22, 2007

(54) Bone screw/driver assembly and method

(75) Inventors: Dennis R. Sullivan JR., Bethany, CT (US); Stephen Santangelo, New Hartford, CT (US); Ernie Corrao, Bethel, CT (US); Stephen Maguire, Shelton, CT (US)

Correspondence Address: HAYNES AND BOONE, LLP, 901 MAIN ST, SUITE 3100, DALLAS, TX, 75202, US

(73) Assignee: Medtronic, Inc., Minneapolis, MN (US)

(21) Appl. No.: 11/196,983

(22) Filed: Aug. 4, 2005

(51) Int. Cl. A61F 2/00

(52) U.S. Cl. 606/104

(57) ABSTRACT

An assembly and method for attaching a medical device, or other structure, to a bone, according to which two projections are formed on one end of a driver that extend in two corresponding recesses, respectively, formed in the head of the screw to drivingly engage the driver and the screw.

### BACKGROUND

The present invention relates to a screw and a driver for driving the screw into a bone.

Self-tapping screws are often needed in surgical procedures to attach a medical device, or other structure, to a bone in the human body. It can be appreciated that when this is done in a surgical procedure, it is important that a driver be provided that can lockingly engage the screw quickly and in a reliable manner to insure that all, or at least substantially all, of the torque applied to the driver is also applied to the screw, without any danger of disengagement between the driver and the screw. The present invention addresses this need.

Various embodiments of the invention may possess one or more of the above features and advantages, or provide one or more solutions to the above problems existing in the prior art.

### BRIEF DESCRIPTION OF THE DRAWINGS

FIG. 1 is an isometric view of a bone screw/driver assembly according to an embodiment.

FIG. 2 is an isometric view of a bone screw/driver assembly according to another embodiment.

FIG. 3 is an exploded isometric/sectional view of the assembly of FIG. 2.

FIGS. 4 and 5 are isometric views of a bone screw/driver assembly according to two additional embodiments.

### DETAILED DESCRIPTION

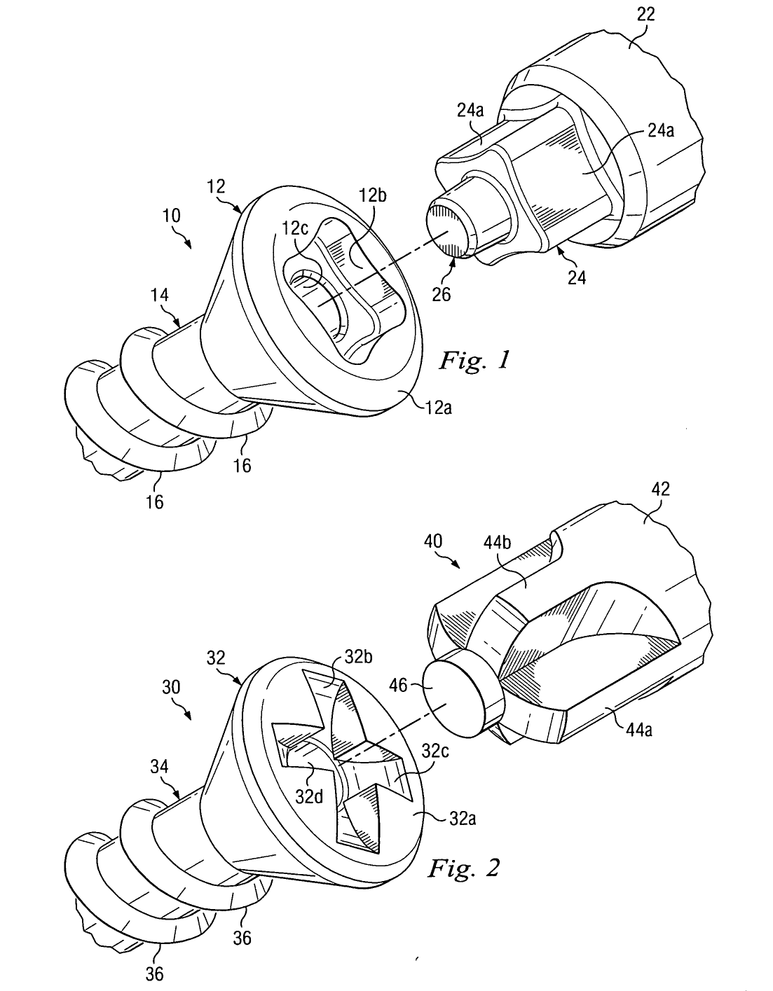

Referring to the embodiment of FIG. 1 of the drawing, the reference numeral 10 refers, in general, to a bone screw comprising a head 12 formed integrally with a shank 14. The head 12 is frusto-conical in cross-section, has a flat distal end surface 12a, and tapers inwardly in a direction from the distal end surface.

The shank 14 is substantially cylindrical in shape and has a helical thread 16 projecting from its outer surface. The screw is “self-tapping”, i.e. when it is driven into a bone, or other structure, by applying torque to the screw, the thread 16 functions to anchor the screw 10 in the bone. It is understood that the distal end portion (not shown) of the shank 14 could be tapered.

A driver is referred to, in general, by the reference numeral 20 and includes a cylindrical handle 22, shown partially. A polygonal projection 24 extends from one end of the handle and is formed integrally therewith. The projection 24 has four walls 24a of substantially the same dimensions to form a substantially square cross-section. Each of the walls 24a is slightly curved to form a concave surface and the width of each wall is less than the diameter of the handle 22.

A cylindrical projection 26 extends from the distal end of the projection 24 and is formed integrally therewith. The diameter of the projection 24 is less than the width of each wall of the projection 24.

A recess 12b is formed in the head 12 of the screw 10, and extends from the flat surface 12a axially into the head. The cross-section of the recess 12b substantially corresponds to the cross-section of the projection 24, with the exception that its corresponding dimensions are slightly greater than the corresponding dimensions of the projection, so as to permit the projection to be inserted into the recess with minimal clearance.

A cylindrical recess, or counterbore, 12c, is also formed in the head 12 and extends from the bottom of the recess 12b further axially into the head. The cross-section of the recess 12c substantially corresponds to the cross-section of the cylindrical projection 26, with the dimensions of the respective cross-sections being such that the projection 26 fits into the recess 12c in an interference fit. (By the term “interference fit” here, and throughout this application, applicants intend to mean the standard engineering definition of the term.)

In use, the driver 20 is placed in operative engagement with the screw 10 by initially inserting the projection 26 through the recess 12b and into the recess 12c, and then rotating the driver 20 relative to the screw 10 or the screw relative to the driver until the projection 24 is in alignment with the recess 12b. The driver 20 is then moved further axially relative to the head 12 until the distal ends of the projections 24 and 26 engage the bottoms of the recesses 12b and 12c, respectively; and/or the flat surface of the end of the handle 22 engages the flat surface 12a of the head 12, to seat the projections in their respective recesses.

The interference fit afforded by the engagement of the projection 26 in the recess 12c enables the surgeon to connect the driver 20 to the screw 10 with one hand if necessary and then locate the end of the shank 14 at the bone. Torque is then applied to the handle 22, either manually or by an electrical device, which causes corresponding rotation of the driver 20 and therefore the screw 10. Axial force is applied to the screw 10 during the application of the torque, causing the screw to be driven into the bone in locking engagement therewith. If a medical device, or other structure, were to be attached to the bone by the screw 10, the screw would initially be inserted through an opening, or the like, in the device or structure before it is driven into the bone in the above manner.

Thus, the assembly of the present invention provides a secure locking engagement between the driver 20 and the screw 10. Also, the driver 20 can engage and drive the screw 10 relatively easily.

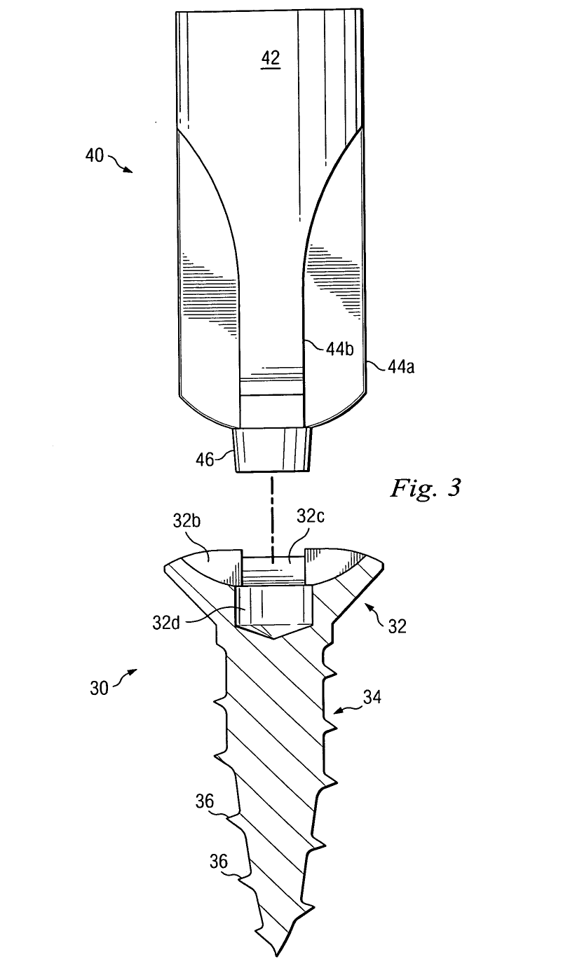

Referring to the embodiment of FIGS. 2 and 3, the reference numeral 30 refers, in general, to a bone screw comprising a head 32 formed integrally with a shank 34. The head 32 is generally frusto-conical in cross-section and tapers inwardly in a direction from the distal end surface.

The shank 34 is substantially cylindrical in shape and has a helical thread 36 projecting from its outer surface. The screw is “self-tapping”, i.e. when it is driven into a bone, or other structure, by applying torque to the screw, the thread 36 functions to anchor the screw 30 in the bone. It is understood that the distal end portion (not shown) of the shank 34 could be tapered.

A driver is referred to, in general, by the reference numeral 40 and includes a cylindrical handle 42, shown partially. Two cross blades 44a and 44b are formed integrally with the handle and extend from one end of the handle. The blades 44a and 44b extend at substantially ninety degrees to each other, and the outer surfaces of the blades are curved to form convex surfaces.

A cylindrical projection, or pin, 46 extends from the centers of the distal ends of the cross blades and is formed integrally therewith. The diameter of the projection 46 is greater than the width of each blade 44a and 44b.

Two recesses, or slots, 32b and 32c extend from the end surface 32a of the head 32 axially into the head and at ninety degrees to each other. The bottoms of the slots 32b and 32c are curved as shown in FIG. 3 in connection with the slots 32b. The cross-section of the slots 32b and 32c substantially correspond to the cross-section of the blades 44a and 44b, with the exception that the dimensions of the slots are very slightly greater than the corresponding dimensions of the blades, so as to permit the blades to be inserted into the slots with minimal clearance.

A cylindrical recess, or counterbore, 32d, is formed in the head 32 and extends from the bottom of the centers of the slots 32b and 32c. The cross-section of the recess 32d substantially corresponds to the cross-section of the projection 46, with the dimensions of their respective cross-sections being such that the projection fits into the recess in an interference fit.

In use, the driver 40 is placed in operative engagement with the screw 30 by initially inserting the projection 46 through the slots 32b and 32c and into the recess 32d, and then rotating the driver 40 relative to the screw 30 until the blades 44a and 44b are in alignment with the slots 32b and 32c. The driver 40 is then moved further axially relative to the head 34 until the distal end of the projection 46 engages the bottom of the recess 32d, and the distal ends of the blades 44a and 44b engage the bottoms of the slots 32b and 32c, to seat the projections and the blades in their respective recesses.

The interference fit afforded by the engagement of the projection 46 in the recess 32d enables the surgeon to connect the driver 40 to the screw 30 with one hand if necessary and then locate the end of the shank 34 at the bone. Torque is then applied to the handle 42, either manually or by an electrical device, which causes corresponding rotation of the driver 40, and therefore the screw 30. Axial force is applied to the screw 30 during the application of the torque, causing the screw to be driven into the bone in locking engagement therewith. If a medical device, or other structure, were to be attached to the bone by the screw 30, the screw would initially be inserted through an opening, or the like, in the device or structure before it is driven into the bone in the above manner.

Thus, the assembly of the embodiment of FIGS. 2 and 3 provides a secure locking engagement between the driver 40 and the screw 30. Also, the driver 40 can engage and drive the screw 30 relatively easily.

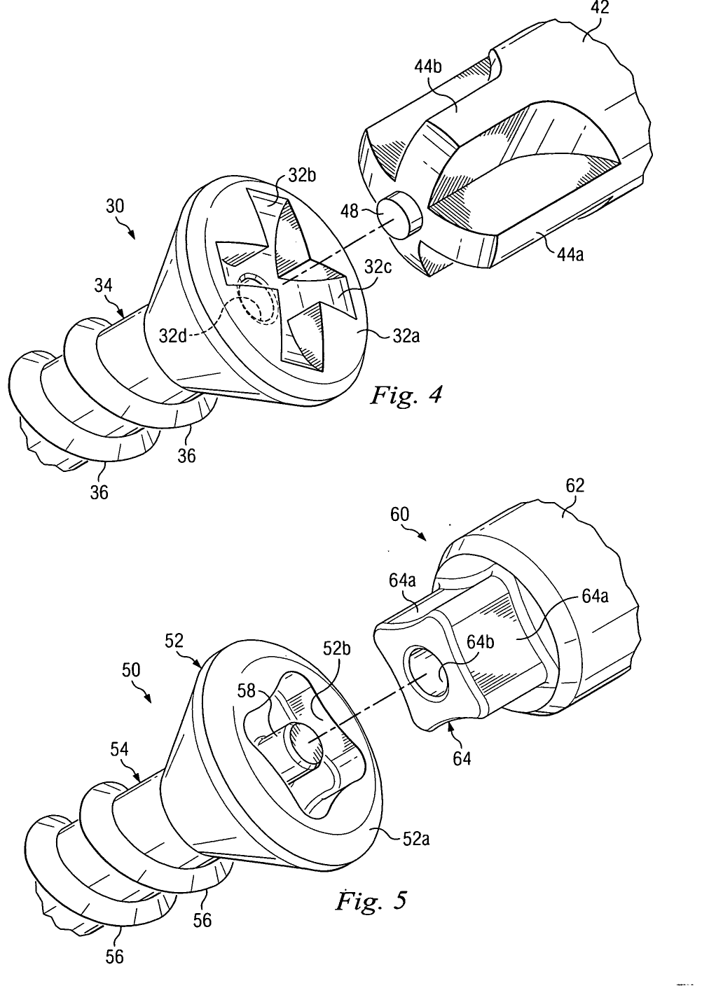

The embodiment of FIG. 4 is similar to that of FIGS. 2 and 3, and includes components that are identical to those of the latter embodiment, which components are given the same reference numerals. According to the embodiment of FIG. 4, the projection, or pin 46 of the embodiment of FIGS. 2 and 3 is replaced by a projection or pin 48 which has a diameter less that the width of the each blade 44a and 44b. Otherwise the embodiment of FIG. 4 is identical to that of FIG. 2.

Referring to the embodiment of FIG. 5, the reference numeral 50 refers, in general, to a bone screw comprising a head 52 formed integrally with a shank 54. The head 52 is frusto-conical in cross-section, has a flat distal end surface 52a, and tapers inwardly in a direction from the distal end surface.

The shank 54 is substantially cylindrical in shape and has a helical thread 56 projecting from its outer surface. The screw is “self-tapping”, i.e. when it is driven into a bone, or other structure, by applying torque to the screw, the thread 56 functions to anchor the screw 50 in the bone. It is understood that the distal end portion (not shown) of the shank 54 could be tapered.

A recess 52b, having a substantially square cross-section, is formed in the head 52 and extends from the flat surface 52a axially into the head. A cylindrical projection 58 projects upwardly from the bottom of the center of the recess 52b. The distal end of the projection 58 extends approximately flush with the end surface 52a, and the diameter of the projection 58 is less than the corresponding dimensions of the recess 52b.

A driver is referred to, in general, by the reference numeral 60 and includes a cylindrical handle 62, shown partially. A polygonal projection 64 extends from one end of the handle and is formed integrally therewith. The projection 64 has four walls 64a of substantially the same dimension to form a substantially square cross-section. Each of the walls 64a is slightly curved to form a concave surface, and the width of each wall is less than the diameter of the handle 62. The cross-section of the projection 64 substantially corresponds to the cross-section of the recess 52b, with the exception that the corresponding dimensions of the projection are slightly less than the corresponding dimensions of the latter recess, so as to permit the projection to be inserted into the recess with minimal clearance.

A cylindrical recess, or counterbore, 64b is formed in the projection 64. The cross-section and depth of the recess 64b substantially corresponds to the cross-section of the cylindrical projection 58 of the screw 50, with the dimensions of the respective cross-sections being such that the projection 58 fits into the recess 64b in an interference fit.

In use, the driver 60 is placed in operative engagement with the screw 50 by initially rotating the driver 60 relative to the screw 50 until the projection 64 is in alignment with the recess 52b. The driver 60 is then moved further axially towards the head 52, causing the projection 64 to enter the recess 52b and the projection 58 to enter the recess 64b. The axial movement is continued until the end of the projection 64 engages the bottom of the recess 52b, and the end of the projection 58 engages the bottom of the recess 64b, to seat the projections into their respective recesses.

The interference fit afforded by the engagement of the projection 58 in the recess 64c enables the surgeon to connect the driver 60 to the screw 50 with one hand if necessary and then locate the end of the shank 54 at the bone. Torque is then applied to the handle 62, either manually or by an electrical device, which causes corresponding rotation of the driver 60, and therefore the screw 50. Axial force is applied to the screw 50 during the application of the torque, causing the screw to be driven into the bone in locking engagement therewith. If a medical device, or other structure, were to be attached to the bone by the screw 50, the screw would initially be inserted through an opening, or the like, in the device or structure before it is driven into the bone in the above manner.

Thus, the assembly of the present invention provides a secure locking engagement between the driver 60 and the screw 50. Also, the driver 60 can engage and drive the screw 50 relatively easily.

### Variations

It is understood that variations may be made in the foregoing without departing from the invention and examples of some variations are as follows:
The design of the shank and/or the threads of each embodiment can be varied.
The dimensions of the projections, and their corresponding recesses in each embodiment can be varied.
The projections 24 and 64 do not have to be perfectly square in cross-section and/or can have more or less than four walls.
The walls of the projections 24 and 64 can be configured in a manner different than that shown in the drawing and described above. For example, one or more of the walls can be curved so as to form a convex surface.
The relative dimensions of the blades 44a and 44b can vary.
The cylindrical projections can be polygonal and vice versa.
The shape of the handles of each embodiment can be varied.
The projections 24 and 64 and the blades 44a and 44b can fit in their corresponding recesses in an interference fit, in which case the projections 26, 46, 48, and 58, would not necessarily fit in their corresponding recesses 12c in an interference fit.
The assembly of the present invention can be used to fasten any type of medical device or structure to a bone.
The spatial references made above are for the purpose of illustration only and do not limit the specific orientation or location of the structure described above.

The preceding specific embodiments are illustrative of the practice of the invention. It is to be understood, therefore, that other expedients known to those skilled in the art or disclosed herein, may be employed without departing from the invention or the scope of the appended claims, as detailed above. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

What is claimed is:

1. An assembly for attaching a medical device, or other structure, to a bone, the device comprising:
a driver comprising:
a body member;
a first projection extending from an end of the body member and having a substantially square cross-section, and
a second projection extending from the first projection; and
a screw comprising:
a head,
a first recess formed in the head and having a cross-section substantially corresponding to the cross-section of the first projection, and
a second recess formed in the head and having a cross-section substantially corresponding to the cross-section of the first projection;
so that when the projections are seated in their respective recesses, the driver and the screw are drivingly connected.

2. The assembly of claim 1 wherein the side walls forming the first projection are curved.

3. The assembly of claim 1 wherein the curves form concave surfaces.

4. The assembly of claim 1 wherein the second projection is cylindrical.

5. The assembly of claim 1 wherein the first recess extends from the surface of the end of the head, and wherein the second recess extends from the bottom of the first recess.

6. The assembly of claim 1 wherein at least one of the projections extends in its corresponding recess in an interference fit.

7. An assembly for attaching a medical device, or other structure, to a bone, the device comprising:
a driver comprising:
a body member,
a first projection extending from an end of the body member, the first projection being polygonal and having at least two curved walls, and
a second projection extending from the first projection; and
a screw comprising:
a head,
a first recess formed in the head and having a cross-section substantially corresponding to the cross-section of the first projection, and
a second recess formed in the head and having a cross-section substantially corresponding to the cross-section of the second projection,
so that when the projections are seated in their respective recesses, the driver and the screw are drivingly connected.

8. The assembly of claim 7 wherein the first projection has four walls of substantially equal width in cross-section to form a substantially square cross-section.

9. The assembly of claim 7 wherein the curved walls form concave surfaces.

10. The assembly of claim 7 wherein the second projection is cylindrical.

11. The assembly of claim 7 wherein the first recess extends from the surface of the end of the head, and wherein the second recess extends from the bottom of the first recess.

12. The assembly of claim 7 wherein at least one of the projections extends in its corresponding recess in an interference fit.

13. An assembly for attaching a medical device, or other structure, to a bone, the device comprising:
a driver comprising:
a body member;
two cross-blades extending from an end of the body member, each cross blade having a substantially curved outer surface, and
a projection projecting from the blades; and
a screw comprising:
a head,
two slots formed in the head and having a cross-section substantially corresponding to the cross section of the blades, and substantially curved bottom surfaces corresponding to the curved outer surfaces of the blades;
a recess formed in the head and having a cross-section substantially corresponding to the cross-section of the projection;
so that when the blades are seated in corresponding slots and the projection is seated in the recess, the driver and the screw are drivingly connected.

14. The assembly of claim 13 wherein the recess in the head extends from the bottoms of the slots.

15. The assembly of claim 13 wherein the slots extend from the surface of the end of the head, and wherein the recess extends from the bottom of the first recess.

16. The assembly of claim 13 wherein the projection extends in the recess in an interference fit.

17. The assembly of claim 13 wherein the projection extends from the center of the distal ends of the cross blades and is formed integrally therewith.

18. An assembly for attaching a medical device, or other structure, to a bone, the device comprising:
a driver comprising:
a body member,
a projection extending from an end of the body member, and
a recess formed in the projection; and
a screw comprising:
a head,
a recess formed in the head and having a cross-section substantially corresponding to the cross-section of the projection, and
a projection extending from the bottom of the recess and having a cross-section substantially corresponding to the cross-section of the recess of the driver,
so that when the projections are seated in their respective recesses, the driver and the screw are drivingly connected.

19. The assembly of claim 18 wherein the first projection of the driver has four walls of substantially equal width in cross-section to form a substantially square cross-section.

20. The assembly of claim 18 wherein the walls are curved.

21. The assembly of claim 18 wherein the projection of the screw is cylindrical.

22. The assembly of claim 18 wherein the recess in the projection of the driver extends from the distal end of the latter projection axially into the latter projection.

23. The assembly of claim 18 wherein recess in the head of the screw extends from the distal end of the head, and wherein the end of the projection in the latter recess extends substantially flush with the distal end of the head.

24. The assembly of claim 18 wherein at least one of the projections extends in its corresponding recess in an interference fit.
